(19) United States
(12) Patent Application Publication
(10) Pub. No.: US 2009/0198146 A1
(43) Pub. Date: Aug. 6, 2009

(54) BLANKING INFECTION MONITORING DURING RECHARGE

(75) Inventors: Martin T. Gerber, Maple Grove, MN (US); John C. Rondoni, Plymouth, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/023,076

(22) Filed: Jan. 31, 2008

(51) Int. Cl. A61B 5/01

(52) U.S. Cl. 600/549

(57) ABSTRACT

A method includes monitoring infection in proximity to a rechargeable implantable medical device; determining whether an event associated with recharging of the device has occurred; and blanking the monitoring if the event has occurred.

### FIELD

This disclosure relates, inter alia, to implantable medical devices. More particularly, it relates to systems, devices and methods for monitoring infection in proximity to medical devices implanted in patients and for blanking the monitoring during recharging of the device.

### BACKGROUND

Infection associated with implantation of medical devices is a serious health and economic concern. Today, infections associated with implanted medical devices are not very common due to care and precautions taken during surgical implantation of the devices. However, when infection associated with an implanted medical device (IMD) does occur, explanting the device is often the only appropriate course of action.

For IMDs having a battery powered component, such as implantable cardiac pacemakers, cardioverter/defibrillators having pacing capabilities, other electrical stimulators including spinal cord, deep brain, nerve, and muscle stimulators, infusion devices, cardiac and other physiologic monitors, cochlear implants, etc., the battery powered component is typically enclosed in a housing that is implanted subcutaneously at a surgically prepared site, referred to as a “pocket”. Associated devices, such as elongated medical electrical leads or drug delivery catheters, extend from the pocket to other subcutaneous sites or deeper into the body to organs or other implantation sites.

Surgical preparation and implantation are conducted in a sterile field, and the IMD components are packaged in sterile containers or sterilized prior to introduction into the sterile field. However, despite these precautions, there always is a risk of introduction of microbes into the pocket. Surgeons therefore typically apply disinfectant or antiseptic agents to the skin at the surgical site prior to surgery, directly to the site before the incision is closed, and prescribe oral antibiotics for the patient to ingest during recovery.

Despite these precautions, infections do occur. In addition, once the pocket becomes infected, the infection can migrate along the lead or catheter to the heart, brain, spinal canal or other location in which the lead or catheter is implanted. Such a migrating infection can become intractable and life-threatening, requiring removal of the IMD in the pocket and associated devices, such as leads and catheters. Removal of a chronically implanted lead or catheter can be difficult and dangerous. Accordingly, aggressive systemic drug treatment is prescribed to treat such infections. However, early detection of infection associated with implanted medical devices may allow for earlier intervention, resulting in fewer device explants.

Monitoring of infection through the use of sensors, such as temperature and pH sensors that can provide information indicative of infection, has been proposed. However, monitoring of infection through sensors connected to an IMD can drain battery power of the IMD. Draining of battery power is not as significant of a concern for rechargeable devices.

However, the recharging process may interfere with the ability to accurately monitor infection. For example, electromagnetic interference noise associated with recharging may cause inaccuracies in infection monitoring circuitry. In addition, localized changes, such as changes in temperature or biological indicators of infection, in tissue surrounding the implanted device or sensors may result from heating or induced power due to recharge, which could result in inaccuracies in infection monitoring. To date, no suggestion has been made to account for inaccuracies that can occur in infection monitoring during recharging of an implantable medical device.

### SUMMARY

The present disclosure describes, inter alia, systems, devices and methods that can be used to monitor an infection in proximity to an implanted medical device, where monitoring of the infection is blanked during an event associated with recharging the device. In an embodiment, a method described herein includes monitoring infection in proximity to a rechargeable implantable medical device; determining whether an event associated with recharging of the device has occurred; and blanking the monitoring if the event has occurred. By providing devices, systems and methods that blank infection monitoring during recharge can provide more reliable monitoring of infection by, for example, not inaccurately identifying infection status while the device is being recharged. Other advantages will be readily understood from the following detailed descriptions when read in conjunction with the accompanying drawings.

### BRIEF DESCRIPTION OF THE DRAWINGS

FIG. 1 is a diagrammatic representation of a perspective view of an environment of a rechargeable system including a rechargeable medical device implanted in a patient.

FIG. 2 is a schematic block diagram of an illustrative rechargeable implantable medical device.

FIG. 3 is a schematic diagram of a side view of a representative implantable medical system.

FIG. 4 is a schematic block diagram of representative components of a rechargeable implantable medical device.

FIGS. 5-9 are flow diagram of representative methods.

The drawings are not necessarily to scale. Like numbers used in the figures refer to like components, steps and the like. However, it will be understood that the use of a number to refer to a component in a given figure is not intended to limit the component in another figure labeled with the same number.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “blank”, “blanking” or the like, as it may relate to infection monitoring, means refraining from performing one or more aspect of infection monitoring. For example, blanking may include refraining from sensing an indicator of infection, refraining from storing sensed information in memory, refraining from determining whether the sensed information is indicative of infection, refraining from issuing an alert if the sensed information is indicative of an infection, or the like.

As used herein “rechargeable medical device” means an implantable medical device having a rechargeable power source, such as a rechargeable battery.

As used herein, “an event associated with recharge” means a detectable stimulus or set of stimuli that is indicative that recharging of a device is occurring or will occur. Examples of such stimuli include characteristic temperature changes in proximity to the device, current or voltage in or across a secondary recharge coil of a device, electrical field strength or electromagnetic interference noise on the secondary coil, and signals from external devices containing information readable by the device that recharge is occurring or will occur.

The present disclosure describes, inter alia, systems, devices and methods that may be used to monitor infection in proximity to an implanted medical device. The systems, devices and methods blank infection monitoring prior to, during or after recharging a power source of an implantable medical device. The blanking can increase the accuracy and reliability of the infection monitoring by, for example, reducing the likelihood of a false positive indication of infection. For example, temperature may be used as an indicator of infection, and recharging an implantable device can increase temperature in proximity to the device. As such, there is a risk that increased temperature due to recharging may be incorrectly interpreted as being due to an infection. Accordingly, blanking infection monitoring during or after recharge may prevent a false positive determination of infection.

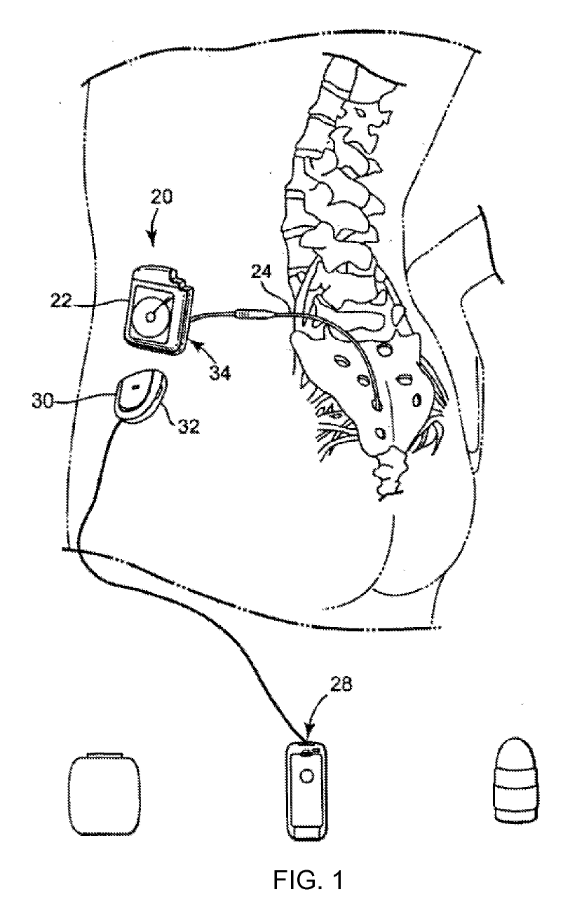

Referring to FIG. 1, a general environment of an embodiment of a representative rechargeable implantable medical device 20 is shown. An implantable electrical signal generator 22 is shown in FIG. 1, but other embodiments such as drug delivery pumps, pacemakers, defibrillators, diagnostic recorders, cochlear implants, monitoring device and the like are also applicable. Implantable medical devices 20 are often implanted subcutaneously approximately one centimeter below the surface of the skin with an associated therapy delivery element, such as an electrical lead 24 or catheter, extending to one or more therapy sites. Rechargeable implantable medical device 20 is recharged with a recharging device 28 such as a patient charger or programmer that also has a charging capability.

Recharging an implantable medical device 20 generally begins with placing a recharging head 30 containing a primary recharging coil 32 against the patient's skin near the proximal side of the medical device 20. Some rechargers 28 have an antenna locator that indicates when recharge head 30 is aligned closely enough with implanted medical device 20 for adequate inductive charge coupling. The recharge power transfer signal is typically a frequency that will penetrate transcutaneous to the location of implanted medical device 20 such as a frequency in the range from 5.0 KHz to 100 KHz. The power transfer signal is converted by implantable medical device 20 into regulated DC power that is used to charge a rechargeable power source 34. Telemetry can also be conducted between the recharger 28 and the implanted medical device 20 during recharging. Telemetry can be used to aid in aligning recharger 28 with the implanted medical device 20, and telemetry can be used to manage the recharging process. Telemetry is typically conducted at a frequency in the range from 150 KHz to 200 KHz using a medical device telemetry protocol, but may also include Bluetooth®, 802.11, and Medical Implant Communication Service (MICS) frequency band communication. For telemetry, the recharger 28 and implanted medical device 20 typically have a separate telemetry coil. Although, the recharging coil can be multiplexed to also serve as a telemetry coil.

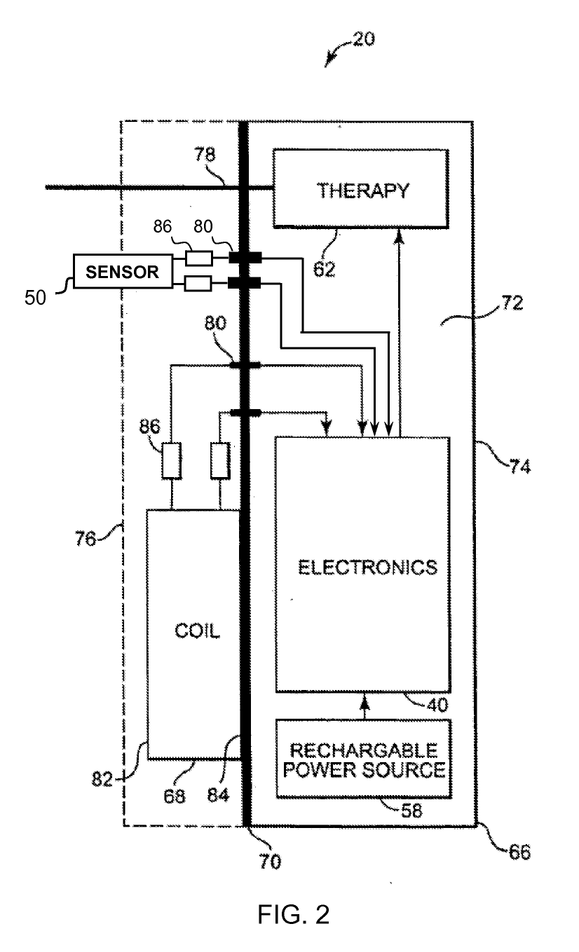

Referring to FIG. 2, a schematic diagram of a representative implantable medical device 20 is shown in block form. Implantable medical device 20 with external recharging coil magnetic shield includes a housing 66, electronics 40, a rechargeable power source 58, a secondary recharging coil 68, and a magnetic shield 70. Housing 66 has an interior cavity 72, an exterior surface 74, a proximal face 76, a therapy connection 78, and a recharge feedthrough 80. The therapy connection 78 can be any type of therapy connection 78 such as a stimulation feedthrough, a drug infusion port, or a physiological sensor. There can also be more than one therapy connection 78 and a combination of different types of therapy connections 78. Housing 66 is hermetically sealed and manufactured from a biocompatible material such as titanium, epoxy, ceramic, and the like. Housing 66 contains electronics 40.

Electronics 40 are carried in the housing interior cavity 72 and, in the embodiment depicted, are configured to perform a medical therapy. Electronics 40 are electrically connected to both a therapy module therapy connection 78 and recharge feedthrough 80. Rechargeable power source 58 is carried in the housing interior cavity 72 and coupled to electronics 40. Rechargeable power source 58 can be a physical power source such as a spring, an electrical power source such as a capacitor, or a chemical power source such as a battery. The battery can be a hermetically sealed rechargeable battery such as a lithium ion (Li+) battery or the like. Electronics 40 are coupled to secondary recharging coil 68.

Secondary recharging coil 68 is coupled to electronics 40 and can also be coupled to rechargeable power source 58 in addition to electronics 40. In various embodiments, the secondary recharging coil 68 can be located on housing proximal face 76, inside housing 66, and remotely away from housing 66. The secondary recharging coil 68 has a proximal side 82 implanted toward a patient's skin and a distal side 84 implanted toward a patient's internal organs. Secondary recharging coil 68 is manufactured from a material with electromagnetic properties such as copper wire, copper magnet wire, copper litz, woven wire, gold alloy or the like. Secondary recharging coil 68 can be manufactured from a wide variety of sizes such as wire diameters in the range from about 0.016 cm (34 AWG, American Wire Gauge) to about 00.40 cm (26 AWG), or any other suitable diameter. Secondary recharging coil 68 is coupled to the recharging feedthroughs 80 with an electrical connection 86. Electrical connection 86 is protected with a hermitic seal to prevent electrical connection 86 from being exposed to biological tissue or fluids. The hermetic seal is a biocompatible material and can take many forms including potting material, polymer encapsulation, coil cover with polymer seal, or the like.

The embodiment depicted in FIG. 2 has secondary recharging coil 68 carried on the proximal face 76 of implantable medical device 20 with magnetic shield 70 positioned between secondary recharging coil 68 and proximal face 76. However, it will be understood that secondary coil 68 may be located at any suitable location, whether within device or external to device 20. External secondary recharging coil 68 increases recharge efficiency because secondary recharging coil 68 is located just under the surface of the skin to decrease coupling distance, and magnetic shield 70 is position to both attract flux lines to the area of secondary recharging coil 68 and reduce flux lines from coupling into housing 66 to reduce eddy currents in housing 66. Additional information regarding recharging of implantable medical devices 20 is provided in U.S. Pat. No. 6,850,803, entitled “Implantable Medical Device With A Recharging Coil Magnetic Shield”, and issued on Feb. 1, 2005; and U.S. patent application Ser. No. 11/737,139, entitled “Controlling Temperature During Recharge for Treatment of Condition”, and filed on Apr. 19, 2007, which application is hereby incorporated by reference in its entirety to the extent that it does not conflict with the disclosure presented herein.

Also shown in the embodiment depicted in FIG. 2, one or more sensors 50 may be coupled to electronics 40. Sensor 50 may be disposed in or on, generally in proximity to, device 20 or portion thereof. Sensor 50 may be exposed to an external surface of device 20 to be in contact with body tissue or fluid when implanted in a patient, or may be contained in housing 66, as appropriate. If sensor 50 is a temperature sensor for monitoring heating of device 20 or surrounding patient tissue during recharge, it may be desirable for sensor 50, 50′ to be located in proximity to secondary coil 68 or near the surface of the device housing 66.

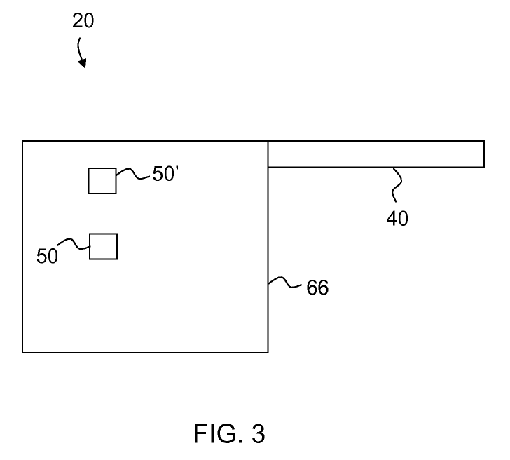

Referring to FIG. 3, a representative rechargeable implantable medical device 20 with an associated therapy delivery element 40 is shown. Therapy delivery element 40 may be a lead 24, catheter, or the like. As shown in FIG. 3, one or more sensors 50, 50′ may be associated with rechargeable implantable medical device. Sensors 50, 50′ may be located in proximity to device 20, e.g. disposed on, in, or near housing 66 of device 20. Sensors 50, 50′ may be used to monitor temperature, an indicator of infection, an indicator of recharge, etc.

In general, sensor 50, 50′ may be any device capable of detecting and transmitting information to device 20. If housing 66 is hermetically sealed, feedthroughs may be used to provide electrical connectivity through housing 66 while maintaining the hermetic seal. While not shown, it will be understood that one or more sensor may be located on, in, or about accessory therapeutic element 40. In various embodiments, (i) sensor 50, 50′ is capable of detecting information regarding an indicator of infection or is capable of detecting and transmitting information that may be useful in determining whether an indicator of infection may actually be indicative of infection, or (ii) sensor 50, 50′ is capable of detecting information regarding an indicator of device recharge.

Additional information regarding sensing, as it relates to infection monitoring, and use of such information in systems including implantable medical devices is provided in (i) U.S. patent application Ser. No. 11/737,180, entitled “INDICATOR METRICS FOR INFECTION MONITORING”, filed on Apr. 19, 2007; and (ii) U.S. patent application Ser. No. 11/737,181, entitled “Multi-Parameter Infection Monitoring”, filed on Apr. 19, 2007, which applications are hereby incorporated herein by reference in their respective entireties to the extent they do not conflict with the disclosure presented herein. Examples of physical or chemical stimuli that may serve as indicators of infection are temperature, impedance, pH, and biological markers of infection. Examples of parameters that may provide information useful for determining whether an indicator of infection may actually be indicative of infection include parameters indicative of patient activity. Examples of stimuli that may serve as an indicator of an event associated with recharging include temperature, current in or voltage across secondary coil 68, electrical field strength or electromagnetic interference on the secondary coil 68, and the like.

In an embodiment, temperature may be used as an indicator of infection and an indicator of recharging. For example, detection of a rapid rise (e.g, over about 30 to 90 minutes) in temperature of about 1° C. may be indicative of recharge, while a more gradual increase in temperature or other characteristic profile may be indicative of an infection. Any suitable sensor 50, 50′ capable of detecting temperature or changes in temperature may be employed. For example, temperature sensor 50, 50′ may include a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, or the like.

The use of more than one temperature sensor at different locations may serve to improve the accuracy of determinations as to whether temperature at a given sensor location is indicative of infection or recharging by comparing the temperature at the given location to temperature at a location removed from the given location. Additional information regarding the use of temperature sensors at two locations for improved infection monitoring is described in U.S. patent application Ser. No. 11/737,171, entitled “Implantable Therapy Delivery System Having Multiple Temperature Sensors”, filed on Apr. 19, 2007, which application is incorporated herein by reference in its entirety to the extent that it does not conflict with the disclosure presented herein. Of course sensor 50′ may detect indicators of infection or recharging or physical or chemical stimuli other than temperature.

Changes in temperature in proximity to implanted device 20 may be used as an indicator of infection in proximity to device 20. The temperature of body tissue at a site of infection is generally greater than that of body tissue at a location removed from the site of infection. Accordingly, an increase in temperature in proximity to an implanted medical device 20 may serve as an indicator of infection.

Changes in impedance of tissue in proximity to implanted device 20 may be used as an indicator of infection in proximity to device 20. For example, an increase in fluid in tissue is often observed at a site of an infection. Accordingly, a decrease in impedance of tissue in proximity may serve as an indicator of infection. In the case of impedance measurement, detection or monitoring, sensors 50, 50′ are electrodes. Impedance may be measured between two electrodes. Current or voltage is applied between the electrode with one electrode at any given time serving as a source and the other serving as a sink. In various embodiments, electrodes will be positioned at opposing surfaces of housing 66 of device 20. In other embodiments, one electrode may be located on accessory device 20, e.g. on a lead, and one may be located on housing of device 20. Alternatively, one electrode may be located on accessory device 40 and housing 66 of device 20 may serve as a return electrode, in a manner similar to unipolar signal generators. Further, it will be understood that more than one electrode pair may be employed to monitor impedance.

In instances where device 20 is an electrical signal generator, the electrical components used for generating therapeutic electrical signals may also be used for generating signals for impedance monitoring. In instances where device 20 is not an electrical signal generator, e.g. device 20 is an infusion pump, components capable of generating appropriate electrical signals for testing impedance of body tissue may be incorporated into device 20. Any impedance detection components or circuitry may be employed. For example, an ohm meter or a wheatstone bridge design may be used to measure or detect changes in impedance or resistance. Examples of additional suitable components or circuitry are described in, for example, the following patents and applications assigned to Medtronic, Inc.: US 2006/0259079; US 2006/0036186; US 2004/0162591; US 2003/0176807; U.S. Pat. No. 5,876,353; U.S. Pat. No. 5,824,029; and U.S. Pat. No. 5,282,840.

Changes in pH in proximity to implanted device 20 may be used as an indicator of infection in proximity to device 20. As pH may serve as a general indicator of the state of a tissue, a change in pH may be indicative of infection. Accordingly, a sudden or gradual change in pH in proximity to an implanted medical device 20 may serve as an indicator of infection. Any suitable sensor 50, 50′ capable of detecting pH or changes in pH may be employed.

Any biological markers of infection may be detected in accordance with the teachings presented herein. Non-limiting examples of biological markers of infection include viral, fungal, or bacterial proteins or nucleic acids or fragments thereof. As most infections associated with implantable medical devices appear to be due to infection due to Staphylococcus aureus, Staphylococcus epidermis, Pseudomonus auruginosa and Candidia Sp., detection of proteins, nucleic acids, or fragments thereof of such microorganisms may be beneficial. Alternatively, detection of indicators of an immune response may be detected. Additional information regarding biological markers of infection are discussed in U.S. patent application Ser. No. 11/737,173, entitled “Infection Monitoring”, and filed on Apr. 19, 2007; and U.S. patent application Ser. No. 11/737,170, entitled “Infection Monitoring”, and filed on Apr. 19, 2007, which applications are hereby incorporated herein by reference in their entireties to the extent they do not conflict with the present disclosure.

Any sensor capable of detecting such biological markers indicative of infection may be used. In various embodiments, a biosensor is used to detect the presence of a molecule in proximity to implanted device 20. Any known or future developed biosensor may be used. The biosensor may have, e.g., an enzyme, an antibody, a receptor, or the like operably coupled to, e.g., a suitable physical transducer capable of converting the biological signal into an electrical signal. In some situations, receptors or enzymes that reversibly bind the molecule being detected may be preferred. In various embodiments, sensor 50, 50′ includes an electrode with an ion selective coating that is capable of directly transducing the amount of a particular substance. An example of this type of transducer is described in the paper “Multichannel semiconductor-based electrodes for in vivo electrochemical and electrophysiological studies in rat CNS” by Craig G. van Home, Spencer Bement, Barry J. Hoffer, and Greg A. Gerhardt, published in Neuroscience Letters, 120 (1990) 249-252. In various embodiments, sensor 50, 50′ may be a sensor as described in, e.g., U.S. Pat. No. 5,978,702, entitled TECHNIQUES OF TREATING EPILEPSY BY BRAIN STIMULATION AND DRUG INFUSION or U.S. 2005/0209513, entitled COLLECTING SLEEP QUALITY INFORMATION VIA A MEDICAL DEVICE, filed Apr. 15, 2004, and published Sep. 22, 2005. Modifications of the teachings presented in the above-cited references may be made to account for one or more biological marker of infection.

For certain biological markers, e.g. proteins or nucleic acids or fragments thereof of microorganisms responsible for infection, merely the presence of such markers may be indicative of an infection. For other markers that may be present in a patient lacking an infection, e.g. cytokines and chemokines, increases or decreases in the levels of such markers may be indicative of an infection.

For the above-discussed indicators of infection or other indicators of infection, a determination of the presence of infection in proximity to implanted device 20 may be made in any suitable fashion. For example, a determination of infection may be made if a given indicator is detected at, above or below a predetermined threshold value. For example, if a temperature of 101° F. (38.3 C) is detected, a determination may be made that an infection is present in proximity to implanted device 20. Alternatively or in addition, a determination of infection may be made if a given indicator is detected at, above or below a predetermined value for a predetermined period of time. For example, if a temperature of 100° F. (37.8 C) or greater is detected for two hours or more is detected for two hours or more, a determination may be made that an infection is present in proximity to implanted device 20. Of course other types of trends in information regarding indicators of infection may be used advantageously to improve the accuracy of determinations of infections in proximity to an implanted medical device.

For the above-discussed indicators of infection or other indicator of infection, it may be desirable to compare levels of the indicators at a location in proximity to device 20 and at a location removed from device. Such comparisons may allow for a reduction in false positive detections. For example, elevation in temperature in proximity to device 20 may be due to localized infection or may be due to increased activity of the patient; increases in inflammatory cytokines in proximity to the device may be due to localized infection or a more general immune response; etc. By comparing the level of an indicator of infection in proximity to an implanted device to the level at a location removed from the device, a more accurate determination of whether an infection is present in proximity to the device may be made.

Information regarding a first indicator of infection may be used to determine whether an infection is present in proximity to the implanted device 20. In addition, one or more second indicators of infection may be used to determine whether the indication based on the first indicator is accurate. Additional information regarding infection monitoring using two or more indicators of infection is provided in U.S. patent application Ser. No. 11/737,181, entitled “Multi-Parameter Infection Monitoring”, filed on Apr. 19, 2007, which application is hereby incorporated herein by reference in its entirety to the extent it does not conflict with the disclosure presented herein.

With regard to detecting recharging of the device 20, temperature may serve as a suitable indicator. Following initiation of recharging temperature at or near device 20 or, more particularly, secondary coil 68 typically increases at a rate more rapid than that associated with infection. For example, temperature of tissue in proximity to device will typically increase by about 1° C. over about 30 to 90 minutes. Such a characteristic temperature profile or other characteristic temperature profiles may be indicative of recharge.

In addition, or alternatively, current or voltage associated with recharge due to coupling of secondary coil 68 with primary coil 32 may be detected by electronics 40. For example, a resistor in the secondary coil 68 circuitry could be used to measure voltage across the resistor. Any appreciable detectable voltage can serve to indicate that recharge is occurring. Sensors capable of detecting electromagnetic field strength or EMI noise at or near the secondary coil 68 may also be employed in recharge detection.

Alternatively, or in addition, a patient programmer, recharger 28, or other external device may send a signal via telemetry or other form of wireless communication that a recharge event is occurring or about to occur. In such instances, programmer, recharger 28 or the like may also send additional instructions regarding blanking infection monitoring, such as which one or more components of monitoring are to be blanked, how long to blank, etc. Such external devices may also communicate with implantable device 20 to indicate that recharge event has ended and that infection monitoring may be resumed.

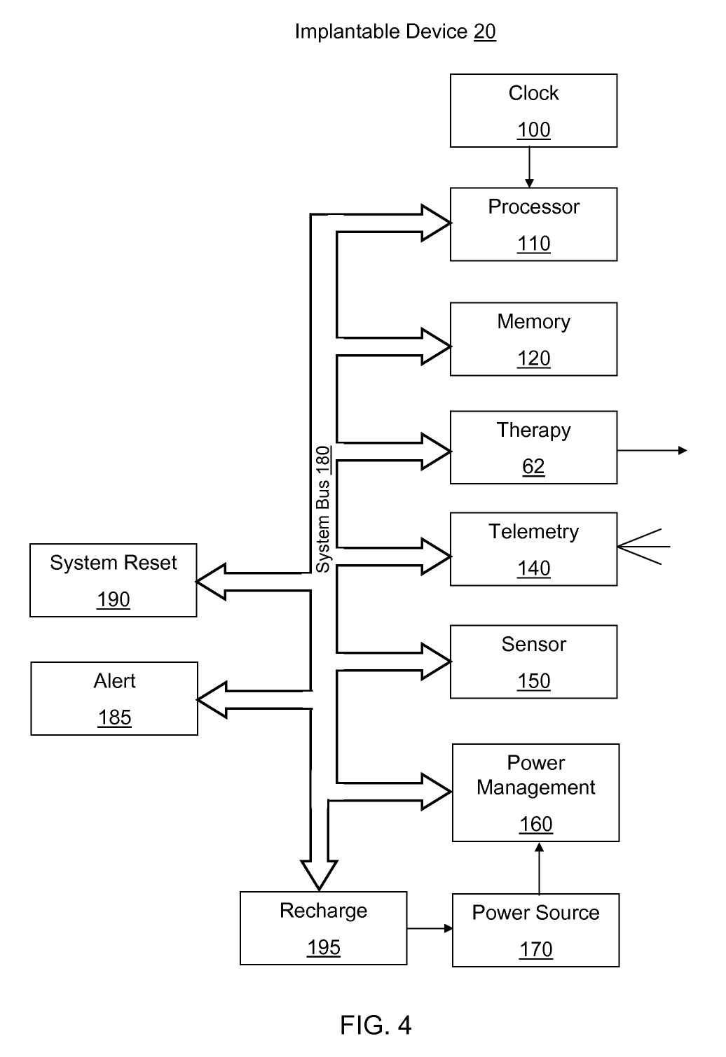

Referring to FIG. 4, some representative electronic components of a rechargeable implantable medical device 20 according to various embodiments are shown in block form. The various components may be contained in, carried on or connected to housing 66. Implantable rechargeable medical device 20 as depicted in the embodiment shown in FIG. 4 includes a clock 100, a processor 110, a memory 120, a therapy output or delivery component 62, a telemetry component 140, a sensor module 150, a power management module 160, a power source 58, an alert module 185, a system reset module 190 and a recharge module 195. Other components of implantable medical device 20 can include, e.g., a diagnostics module (not shown). In the embodiment depicted in FIG. 4, all components except the power source 58 can be configured on one or more Application Specific Integrated Circuits (ASICS) or may be one or more discrete components, or a combination of both. Also, all components, except the clock and power source may be connected to bi-directional data bus 180 that is non-multiplexed with separate address and data lines.

Processor 110 may be synchronous and typically operates on low power, such as Motorola 68HC11 synthesized core operating with a compatible instruction set. Clock 100 counts the number of seconds since a fixed date for date/time stamping of events and may be used for therapy control. Memory 120 includes memory sufficient for operation of device 1, such as volatile Random Access Memory (RAM) for example static RAM, nonvolatile Read Only Memory (ROM), Electrically Erasable Programmable Read Only Memory (EEPROM) for example Flash EEPROM, and register arrays configured on ASICs. Direct Memory Access (DMA) is available to selected modules such as telemetry module 6 or sensor module 150, so that the selected modules can request control of data bus 180 and write data directly to memory 120 bypassing processor 110. System Reset 190 controls operation of ASICs and modules during power-up of device 20, so ASICs and modules registers can be loaded and brought on-line in a stable condition.

Telemetry 140 module or other wireless module provides for communication between implantable device 20 and external device 40 such as a programmer or a recharger 28. Communication may be bi-directional. Telemetry module 140 generally includes a telemetry antenna, a receiver, a transmitter, and a telemetry processor. In some embodiments, a recharge coil may be co-opted for use as a telemetry antenna. Telemetry modules are generally known in the art and are further detailed in U.S. Pat. No. 5,752,977, entitled “Efficient High Data Rate Telemetry Format For Implanted Medical Device” issued to Grevious et al. (May 19, 1998), which is incorporate herein by reference in its entirety to the extent that it does not conflict with the disclosure presented herein. While module 140 is referred to herein as “telemetry” module, it will be understood that other forms of wireless communication may readily be substituted where appropriate for telemetry. Examples of forms of wireless communication include Bluetooth®, 802.11, and Medical Implant Communication Service (MICS) frequency band communication.

Therapy module 62 refers to components for carrying out the delivery or generation of therapeutic output to be delivered to a patient from active device 20. One of skill in the art will appreciate that the components may vary on a device-by-device basis and a therapy-by-therapy basis. For example, therapy module 62 may contain an oscillator if device 20 is an electrical signal generator and may contain a pumping mechanism if device 20 is an infusion device.

Sensor module 150 includes circuitry associated with one or more sensors 50, 50′ and may include other components for transmitting sensed information from sensor 50, 50′ to, e.g., processor 110 or memory 120. Sensor module 150 or other components of device 20 may include one or more analog to digital converters to convert analog signals generated by sensor 50 into digital signals usable by processor 110, as well as suitable filter and amplifier circuitry.

Alert module 185 may issue an alert, e.g. an audible alert or tactile alert, such as a vibration. An alert may be issued if information indicative of an infection is detected, if a potential adverse situation, e.g. excessive heating of device 20, is detected, if a power source is nearing depletion, or the like. The alert may serve to prompt the patient to seek medical attention.

It may be desirable in some circumstances for hardware resources of recharge module 195 to dedicated to the recharge process and not be multiplexed into infection monitoring processes to prevent interference or interruption of the recharge process by the infection monitoring process.

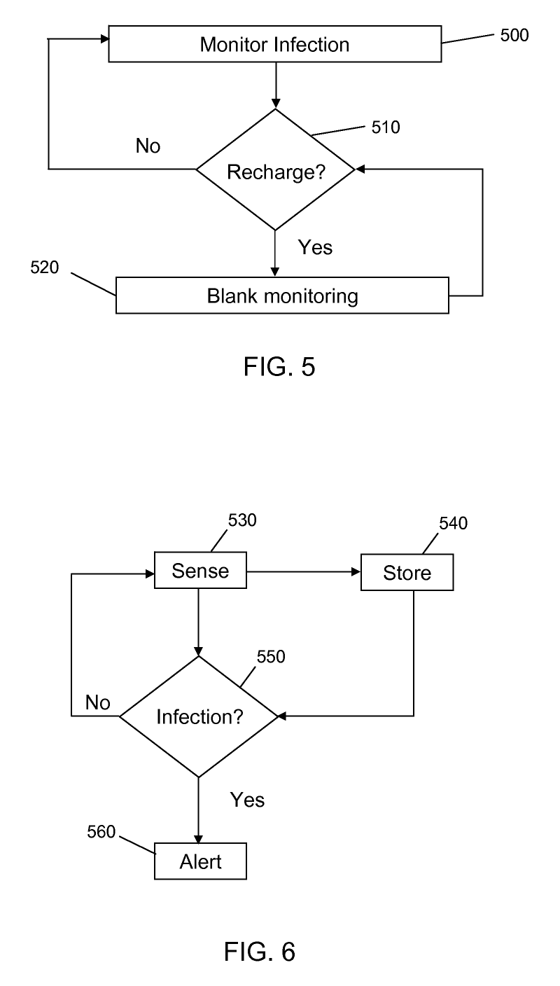

It will be understood that the components described in FIGS. 1-4 are but examples of components that an implantable device 20 or an associated system may include and that many other device or system configurations may be employed to carry out the methods described below. However, for the sake of convenience, the discussion that follows with regard to the methods illustrated in the flow diagrams of FIGS. 5-9 will refer to components as described with regard to FIGS. 1-4.

Referring to FIG. 5, a flow diagram of a representative method is shown. According to various embodiments, a method for monitoring infection and blanking during recharge includes monitoring an indicator of infection in proximity to a rechargeable implantable medical device 20 (500) and determining whether an event associated with recharging of the device 20 has occurred (510). If an event associated with recharge has occurred, the monitoring is blanked (520).

With reference to FIG. 6, monitoring infection in proximity to an implantable medical device (500) may include one or more of (i) sensing (530) one or more indicator of infection, (ii) storing information regarding the sensed indicator (540), (iii) determining whether information regarding the sensed indicator is, or combination of sensed indicators are, indicative of an infection (550), and issuing an alert if the if the sensed indicator(s) is indicative of infection (560). A determination as to whether the sensed information is indicative of infection (550) may be made by processor 110 based on information as it is received from sensor module 150. Alternatively, or in addition, sensed information may be stored (500) in memory 120, and processor 110 may retrieve sensed information stored in memory 120 to determine whether the information is indicative of infection. While not shown, it will be understood that the determination as to whether the sensed information is indicative of infection (550) may be made by an external device. For example, sensed information, whether stored in memory 120 or as received from sensor module 150 may be transmitted to an external device via telemetry module 140 for the determination (550) to be made. If a determination is made that sensed information is indicative of infection, an alert may be issued (560). For example, processor 110 may activate alert module 185 to prompt patient to seek medical attention. As with the determination (550), the alert (560) may be issued by the implantable medical device 20 or by an external device.

A determination as to whether an event associated with recharge has occurred (510) may include use of nearly any suitable stimuli. Examples of suitable stimuli include temperature changes in proximity to or generally within device 20, changes in electrical properties associated with a recharge coil 68, signals provided by external devices such as a programmer or recharger 28. For example, one or more temperature sensors 50, 50′ may be employed to transmit information regarding temperature to electronics 40 to determine whether a characteristic change in temperature has occurred, whether an absolute temperature threshold has been met or exceeded, or the like. By way of further example, electrical or electromagnetic properties of at or near the secondary recharge coil 68 that are indicative of a recharge event may be detected by electronics 40 to determine that a recharge event has occurred. By way of yet another example, a signal from an external device may be received by telemetry module 140 and transmitted to processor 110 to determine whether a recharge event has occurred.

It will be understood some events may occur over a period of time and that detection of an event that “has occurred” or a determination of whether an event “has occurred” may include detection of events that are occurring. For example, recharging a power source 170 of a device 20 typically occurs over a period of time. However, a characteristic temperature profile may be associated with the initial phase of recharge. The detection of such a characteristic initial phase temperature profile may result in a determination that a recharge event (i.e., initiation of recharge) “has occurred” even though overall recharge event may be still occurring or ongoing. By way of further example, a signal sent by an external device indicating that a recharge will occur in the future is an event associated with recharging the device.

In various embodiments, information received from sensor module 150, recharge module 195, or telemetry module 140 is transmitted to processor 110 either directly or via memory 120 so that processor 110 may determine whether an event associated with recharge has occurred. If a recharge event is determined to have occurred, infection monitoring is blanked (520).

Blanking of infection monitoring (520) may occur by refraining from performing one or more aspects of infection monitoring (500). For example, blanking infection monitoring (520) may include refraining from (i) sensing (530) an indicator of infection, (ii) storing sensed information (540), (iii) determining whether the sensed information is indicative of infection (550), or (iv) issuing an alert (560). In various embodiments, refraining from (i) sensing (530), (ii) storing (540), or (iii) determining (550) will result in an alert not being issued, which can prevent a false positive alert and prevent unnecessary concern for the patient in which the device is implanted. In various embodiments, a determination as to whether a recharge event has occurred (510) is made by processor 110, which can instruct sensor module 150 to refrain from sensing an indicator of infection, which will effectively blank infection monitoring (520). In addition or alternatively, processor 110 may refrain from making a determination as to whether an infection has occurred (550) once a determination has been made that an event associated with recharge has occurred (510) or may instruct alert module 185 to refrain from issuing an alert.

It may be desirable to store information regarding sensed indicators of infection (540), and thus continue to sense the indicators (530), during a period of blanking. By continuing to store such information during a period of blanking, the effects of recharge on such information may later be determined. In various embodiments, following a determination that a recharge event has occurred (510), processor 110 time stamps the event via information received from clock module 100. Processor 110 may also time stamp the end of recharging of the device (discussed in more detail below). Stored information regarding the sensed indicator during the time of recharge may later be analyzed to determine whether any false positive determinations of infection would have occurred or to determine whether certain characteristic patterns can be elucidated and later corrected for so that accurate infection monitoring may continue during periods of recharge. Additional relevant information may be taken also be taken into account. For example, the level of battery depletion may have an effect on the duration of the recharge, the electrical parameters associated with recharge, the temperature change as a result of the recharge and the like. Strength of coupling between the primary coil 32 in the recharge head 30 and the secondary coil 68 may also affect similar parameters. These and other parameters may also be taken into account to develop appropriate algorithms to correct for the effects of recharge on infection monitoring so that infection monitoring may continue as the device is being recharged.

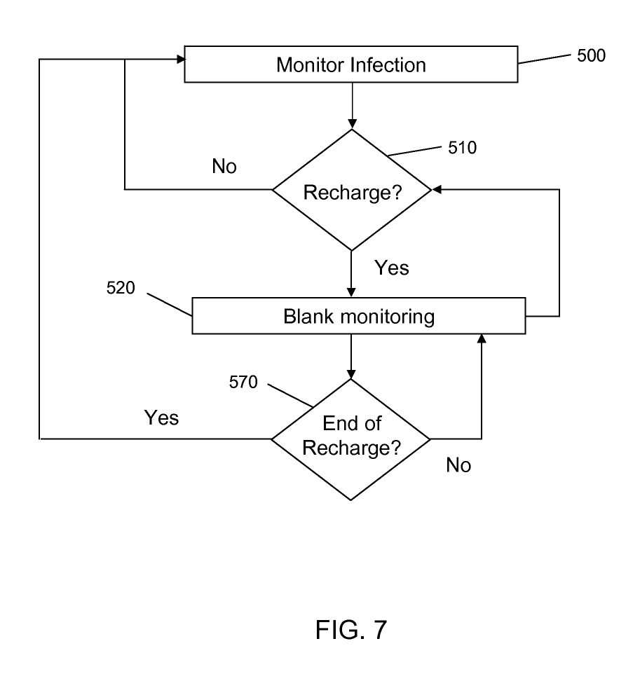

Following recharge, it may be desirable to resume all aspects of monitoring infection (500). Referring to FIG. 7, a determination may be made as to whether the end of recharging has occurred (570). If it is determined that the end of recharge has occurred, infection monitoring is resumed (500). Otherwise, blanking of monitoring (520) continues. The end of a recharge event may be detected in a similar manner to the initiation of a recharge event. For example, a characteristic temperature decrease, a decrease in current or voltage on the secondary recharge coil 68, or a signal sent via telemetry from an external device may be indicative of the end of recharge. In various embodiments, sensor module 150 transmits to processor 110 information regarding temperature, and processor 110 determines whether the sensed temperature information is indicative of the end of a recharge event; e.g. whether a characteristic decrease in temperature is detected. In various embodiments, telemetry module 140 transmits information regarding a signal received from an external device to processor 110, which then determines whether the signal is indicative of an end of recharge. In various embodiments, recharge module 195 sends information to processor 110 regarding the amount of current or voltage on secondary recharge coil 68. Processor 110 may then determine whether such information is indicative of an end of recharge.

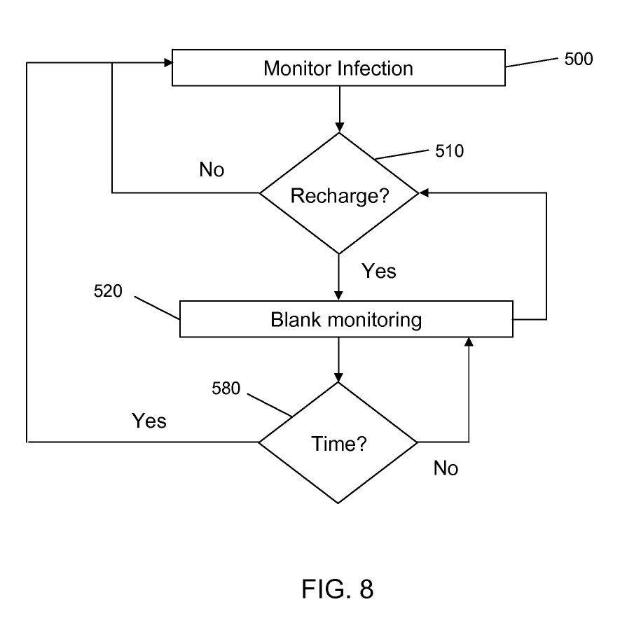

Referring to FIG. 8, as an alternative to determining whether the end of recharge has occurred, monitoring of infection (500) may be resumed after a predetermined period of time has elapsed (580). The amount of time may be the amount of time for a typical recharge. In various embodiments, blanking of infection monitoring (520) continues for a period of time for a typical recharge plus an additional amount of time as a buffer. The buffer time may be sufficiently long to avoid accidental resumption of infection monitoring (500) during an abnormally long recharge or may be sufficient time to allow conditions to return substantially to baseline relative to prior to recharge; e.g., to allow temperature in proximity to device to return to a temperature prior to initiation of recharging. The determination as to whether sufficient time has elapsed (580) may be made by processor 110. For example, upon determination that a recharge event has occurred (510), processor 110 may time stamp the event via information received from clock module 100. Once a determination has been made by processor 110 that sufficient amount of time has elapsed (580), processor 110 may instruct the appropriate module(s) to resume infection monitoring (500). In various embodiments, instructions regarding the timing of the end of recharge may be provided by recharger 28 in conjunction with or following sending a signal indicative of initiation of a recharge event.

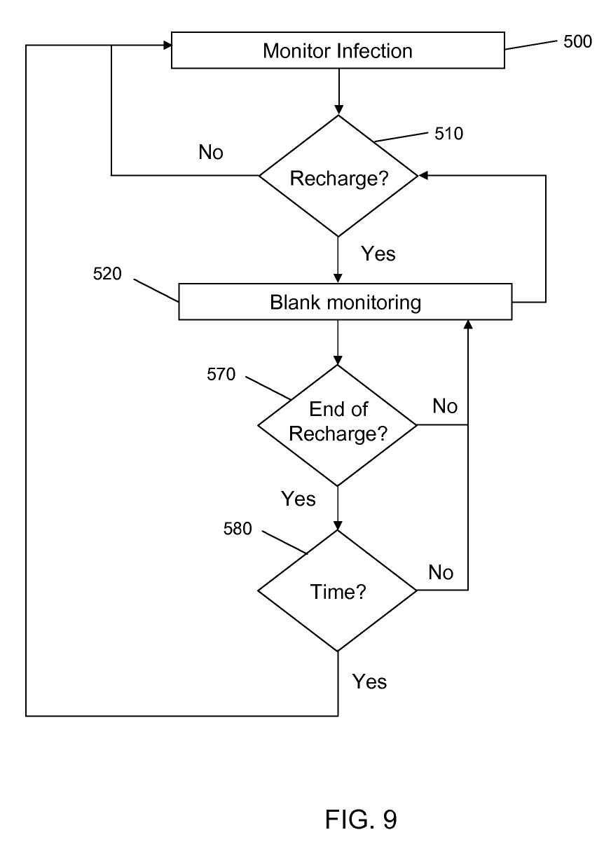

Referring to FIG. 9, in which a flow diagram of a representative method is shown, a determination as to whether recharging has ended is made (570) in addition to waiting a predetermined period of time (580) prior to fully resuming infection monitoring (500). Such a method allows for conditions to return to baseline prior to resuming infection monitoring to further improving the accuracy of the monitoring.

One of skill in the art will understand that components or steps described herein regarding a given embodiment or set of embodiments may readily be omitted, substituted, or added from, with, or to components or steps of other embodiments or sets of embodiments, as appropriate or desirable.

It will be further understood that a computer readable medium containing instructions that when implemented cause an implantable medical device (or system including an implantable medical device) to perform the methods described herein are contemplated. In an embodiment the computer readable medium contains instructions that when implemented cause an implantable medical device to (i) monitor infection in proximity to the device, (ii) detect an event associated with recharging of the device; and (iii) blank the monitoring during recharging of the device. Devices including the computer readable medium are also contemplated.

Patent applications that may provide additional insight into the teachings provided herein include the following: (i) U.S. patent application Ser. No. 11/737,176, entitled “Refined Infection Monitoring”, filed on Apr. 19, 2007; (ii) U.S. patent application Ser. No. 11/737,169, entitled “Event Triggered Infection Monitoring”, filed on Apr. 17, 2007; (iii) U.S. Provisional Application Ser. No. 60/912,078, entitled “Heating Implantable Device to Treat a Condition”, filed on Apr. 19, 2007; and (iv) U.S. patent application Ser. No. ______, entitled “Baseline Acquisition ofr Infection Monitoring”, filed on even date herewith, naming Martin Gerber and John Rondoni as inventors, and having attorney docket number P0029356.00. Each of the above-referenced patent applications is hereby incorporated herein by reference in their respective entireties to the extent that they do not conflict with the disclosure presented herein.

Thus, embodiments of BLANKING INFECTION MONITORING DURING RECHARGE are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method performed by an implantable medical device, comprising:
monitoring infection in proximity to a rechargeable implantable medical device;
determining whether an event associated with recharging of the device has occurred; and
blanking the monitoring if the event has occurred.

2. The method of claim 1, wherein monitoring infection comprises temperature.

3. The method of claim 1, wherein monitoring infection comprises one or more of sensing an indicator of infection, storing information regarding the sensed indicator, determining whether information regarding the sensed indicator is indicative of an infection, and issuing an alert if the if the sensed indicator is indicative of infection.

4. The method of claim 3, wherein blanking comprises refraining from issuing the alert.

5. The method of claim 3, wherein blanking comprises refraining from determining whether information regarding the sensed indicator is indicative of an infection.

6. The method of claim 3, wherein blanking comprises refraining from storing information regarding the sensed indicator.

7. The method of claim 3, wherein blanking comprises refraining from sensing information regarding the indicator.

8. The method of claim 1, wherein determining whether the event has occurred comprises receiving a signal from an external device.

9. The method of claim 8, wherein determining whether the event has occurred comprises detecting a change in temperature.

10. The method of claim 8, wherein determining whether the event has occurred comprises detecting current in a secondary recharge coil of the device.

11. The method of claim 1, further comprising reinitiating infection monitoring following the blanking.

12. The method of claim 11, wherein the reinitiating comprises resuming monitoring after completion of the recharging of the device.

13. The method of claim 12, further comprising detecting the completion of the recharging of the device.

14. The method of claim 13, wherein detecting the completion of the recharging of the device comprises receiving a signal from an external device.

15. The method of claim 13, wherein detecting the completion of the recharging of the device comprises detecting a change in temperature.

16. The method of claim 13, wherein detecting the completion of the recharging of the device comprises detecting current in a secondary recharge coil of the device.

17. The method of claim 13, further comprising determining whether a predetermined amount of time has elapsed prior to resuming the infection monitoring.

18. A computer readable medium containing instructions that when implemented cause an implantable rechargeable medical device to:
monitor infection in proximity to the device,
detect an event associated with recharging of the device; and
blank the monitoring during recharging of the device.

19. An implantable medical device comprising:
a rechargeable power source;
electronics operably coupled to the power source and configured to monitor an infection in proximity to the device;
a sensor operably coupled to the electronics and capable of monitoring an indicator of infection;
a computer readable medium according to claim 18 readable and executable by the electronics.
